(19) United States
(12) Patent Application Publication
(10) Pub. No.: US 2025/0057653 A1
(43) Pub. Date: Feb. 20, 2025

(54) TRANSCATHETER PROSTHETIC HEART VALVE DELIVERY SYSTEMS AND METHODS OF USE

(71) Applicant: Medtronic, Inc., Minneapolis, MN (US)

(72) Inventors: Matthew P. Norgrove, Dublin (IE); Nicholas Fox, Oranmore (IE)

(21) Appl. No.: 18/723,356

(22) PCT Filed: Feb. 14, 2023
(86) PCT No.: PCT/IB2023/051326

§ 371 Date: Jun. 21, 2024

Related U.S. Application Data
(60) Provisional application No. 63/312,866, filed on Feb. 23, 2022.

(51) Int. Cl. A61F 2/24

(52) U.S. Cl. CPC A61F 2/2436; A61F 2/2433

(57) ABSTRACT

In some embodiments, a prosthetic heart valve delivery device is provided that includes an outer shaft received over an inner shaft, and rod. The rod is selectively inserted or advanced along a slot in the outer shaft to increase a torqueability of the outer shaft. In some embodiments, a prosthetic heart valve delivery device is provided that includes a handle assembly, an outer shaft received over an inner shaft, a stability shaft received over the outer shaft, and a wire. A leading section of the wire is affixed to the stability shaft, and a trailing section of the wire extends proximally from the stability shaft and is selectively engaged by a locking mechanism of the handle assembly. In a locked state of the locking mechanism, tension is maintained in the wire and generates a bending stiffness in the stability shaft.

### FIELD

The present disclosure relates to catheter-based devices and systems for delivering a prosthetic heart valve. More particularly, it relates to transcatheter prosthetic heart valve delivery devices and corresponding methods of use.

### BACKGROUND

A human heart includes four heart valves that determine the pathway of blood flow through the heart: the mitral valve, the tricuspid valve, the aortic valve, and the pulmonary valve. The mitral and tricuspid valves are atrio-ventricular valves, which are between the atria and the ventricles, while the aortic and pulmonary valves are semilunar valves, which are in the arteries leaving the heart. Ideally, native leaflets of a heart valve move apart from each other when the valve is in an open position, and meet or “coapt” when the valve is in a closed position. Problems that may develop with valves include stenosis in which a valve does not open properly, and/or insufficiency or regurgitation in which a valve does not close properly. Stenosis and insufficiency may occur concomitantly in the same valve. The effects of valvular dysfunction vary, with regurgitation or backflow typically having relatively severe physiological consequences to the patient.

Diseased or otherwise deficient heart valves can be repaired or replaced using a variety of different types of heart valve surgeries. One conventional technique involves an open-heart surgical approach that is conducted under general anesthesia, during which the heart is stopped and blood flow is controlled by a heart-lung bypass machine.

More recently, minimally invasive approaches have been developed to facilitate catheter-based implantation of the valve prosthesis on the beating heart, intending to obviate the need for the use of classical sternotomy and cardiopulmonary bypass. In general terms, an expandable prosthetic valve is compressed about or within a catheter, inserted inside a body lumen of the patient, such as the femoral artery, and delivered to a desired location in the heart.

The heart valve prosthesis employed with catheter-based, or transcatheter, procedures generally includes an expandable multi-level frame or stent that supports a valve structure having a plurality of leaflets. The frame can be contracted during percutaneous transluminal delivery, and expanded upon deployment at or within the native valve. One type of valve stent can be initially provided in an expanded or uncrimped condition, then crimped or compressed about a balloon portion of a catheter. The balloon is subsequently inflated to expand and deploy the prosthetic heart valve. With other stented prosthetic heart valve designs, the stent frame is formed to be self-expanding. With these systems, the valved stent is crimped down to a desired size and held in that compressed state within a sheath for transluminal delivery. Retracting the sheath from this valved stent allows the stent to self-expand to a larger diameter, fixating at the native valve site. In more general terms, then, once the prosthetic valve is positioned at the treatment site, for instance within an incompetent native valve, the stent frame structure may be expanded to hold the prosthetic valve firmly in place. One example of a stented prosthetic valve is disclosed in U.S. Pat. No. 5,957,949 to Leonhardt et al., which is incorporated by reference herein in its entirety.

The present disclosure addresses problems and limitations associated with the related art.

### SUMMARY

Some aspects of the present disclosure are directed to a transcatheter prosthetic heart valve delivery device. The delivery device includes an outer shaft assembly, an inner shaft assembly and a rod. The outer shaft assembly defines a longitudinal axis, a proximal end, and a distal end opposite the proximal end. The outer shaft assembly further defines a central lumen and a slot. The slot extends through a thickness of a side wall of the outer shaft assembly, and is open at the proximal end. The inner shaft assembly is configured to be co-axially received within the central lumen of the outer shaft assembly. The rod is sized to be slidably received within the slot, and defines an upper surface opposite a lower surface. A thickness of the rod between the upper and lower surfaces varies along a length of the rod. The rod is selectively inserted into the slot via the proximal end such that the upper surface is radially opposite the lower surface relative to the longitudinal axis. In some embodiments, the transcatheter prosthetic heart valve delivery device is configured such that insertion of the rod into the slot increases a torqueability of the outer shaft assembly. In some embodiments, the rod defines a corrugated shape along the length thereof. In some embodiments, the delivery device further includes a handle assembly maintaining the inner and outer shaft assemblies. The handle assembly includes a locking mechanism configured to selectively engage the rod to longitudinally lock the rod relative to the outer shaft assembly.

Other aspects of the present disclosure are directed to a method of delivering a prosthetic heart valve to a target site. The method includes receiving a delivery device loaded with a prosthetic heart valve in a compressed state. The delivery device includes an outer shaft assembly and an inner shaft assembly. The outer shaft assembly defines a longitudinal axis, a proximal end, and a distal end opposite the proximal end. The outer shaft assembly further defines a central lumen and a slot. The slot extends through a thickness of a side wall of the outer shaft assembly, and is open at the proximal end. The inner shaft assembly is configured to be co-axially received within the central lumen of the outer shaft assembly and carries the prosthetic heart valve. The delivery device is advanced through a vasculature of a patient such that the distal end approaches a target site with the distal end at a first rotational arrangement relative to the target site. A rod is advanced within the slot while the delivery device is at the first rotational arrangement. A torque is applied onto the outer shaft assembly to rotate the delivery device from the first rotational arrangement to a second rotational arrangement relative to the target site while the rod remains advanced within the slot. Presence of the rod within the slot facilitates rotation of the distal end in response to the applied torque. The delivery device to deploy the prosthetic heart valve at the target site. In some embodiments, the step of advancing the delivery device includes directing the distal end along and beyond an aortic arch of the patient; further, the step of advancing the rod occurs after the step of advancing the delivery device and includes directing a first end of the rod along and beyond the aortic arch.

Other aspects of the present disclosure are directed to a transcatheter prosthetic heart valve delivery device. The delivery device includes a handle assembly, an outer shaft assembly, an inner shaft assembly, a stability shaft and a wire. The handle assembly includes a locking mechanism. The outer shaft assembly extends from the handle assembly and defines a central lumen. The outer shaft assembly includes a distal region and a proximal region. The inner shaft assembly extends from the handle assembly and is configured to be co-axially received within the central lumen. The stability shaft extends from the handle assembly and defines a central passage sized to slidably receive the proximal region of the outer shaft assembly. The stability shaft terminates at a distal end opposite the handle assembly. The wire defines a leading section opposite a trailing section. The leading section is affixed to the stability shaft proximate the distal end. The trailing section extends proximally beyond the stability shaft and is arranged to be selectively engaged by the locking mechanism. The locking mechanism is operable to selectively lock the trailing section relative to the handle assembly such that the delivery device provides an unlocked state in which the trailing section freely slides relative to the handle assembly and a locked state in which the trailing section is locked relative to the handle assembly to maintain tension in the wire. In some embodiments, the delivery device is configured such that in the locked state, tension in the wire generates a bending stiffness in the stability shaft.

Other aspects of the present disclosure are directed to a method of delivering a prosthetic heart valve to a target site. The method includes receiving a delivery device loaded with a prosthetic heart valve in a compressed state. The delivery device includes a handle assembly, an outer shaft assembly, an inner shaft assembly, a stability shaft and a wire. The handle assembly includes a locking mechanism. The outer shaft assembly extends from the handle assembly and defines a central lumen. The outer shaft assembly includes a distal region and a proximal region. The inner shaft assembly extends from the handle assembly and is configured to be co-axially received within the central lumen. The stability shaft extends from the handle assembly and defines a central passage sized to slidably receive the proximal region of the outer shaft assembly. The stability shaft terminates at a distal end opposite the handle assembly. The wire defines a leading section opposite a trailing section. The leading section is affixed to the stability shaft proximate the distal end. The trailing section extends proximally beyond the stability shaft and is arranged to be selectively engaged by the locking mechanism. The prosthetic heart valve is disposed over the inner shaft assembly and is contained within a capsule of the outer shaft assembly. The delivery system is advanced through a vasculature of a patient with the locking mechanism in an unlocked state such that the trailing section of the wire freely slides relative to the handle assembly during the step of advancing. The locking mechanism is transitioned from the unlocked state to a locked state such that the trailing section is locked relative to the handle assembly and tension in the wire is maintained. The outer shaft assembly is retracted relative to the inner shaft assembly and relative to the stability shaft to deploy the prosthetic heart valve. In this regard, the locking mechanism remains in the locked state during the step of proximally retracting. In some embodiments, tension in the wire generates an increased bending stiffness in the stability shaft.

### BRIEF DESCRIPTION OF DRAWINGS

FIG. 1 is an exploded, perspective view of a transcatheter prosthetic heart valve delivery device in accordance with principles of the present disclosure;

FIG. 2 is a cross-sectional view of an outer shaft assembly useful with the delivery device of FIG. 1;

FIG. 3 is a perspective view of a portion of a rod useful with the delivery device of FIG. 1;

FIG. 4A is a side view of the rod of FIG. 3;

FIG. 4B is a top view of the rod of FIG. 3;

FIG. 5A illustrates a response of the rod of FIG. 3 to the forces shown in FIG. 4A;

FIG. 5B illustrates a response of the rod of FIG. 3 to the forces shown in FIG. 4B;

FIG. 6 is a cross-sectional view of a portion of the delivery device of FIG. 1, including the rod of FIG. 3 assembled to the outer shaft assembly of FIG. 2;

FIG. 7A illustrates, in simplified form, portions of the delivery device of FIG. 1, including a rod inserted within an outer shaft assembly, and transmission of torque along the outer shaft assembly;

FIG. 7B illustrates, in simplified form, the outer shaft assembly and rod of FIG. 7A, and flexibility of the rod in conforming to bends in the outer shaft assembly;

FIG. 8A is a simplified side view of portions of the delivery device of FIG. 1, including an outer shaft assembly maintained by a handle assembly;

FIG. 8B is a simplified side view of portions of the delivery device of FIG. 1, including a rod assembled to the outer shaft assembly and handle assembly of FIG. 8A;

FIG. 9A is a simplified cross-sectional view of a prosthetic heart valve mounted to a delivery device useful as the delivery device of FIG. 1;

FIG. 9B is a simplified side view of a prosthetic heart prosthetic heart valve mounted to portions of a delivery device useful as the delivery device of FIG. 1;

FIG. 10A-10C illustrate use of the delivery device of FIG. 1 in percutaneously delivering a prosthetic heart valve to a target site in accordance with methods of the present disclosure;

FIG. 11 is an exploded, perspective view of a transcatheter prosthetic heart valve delivery device in accordance with principles of the present disclosure;

FIG. 12 is a cross-sectional view of a stability shaft useful with the delivery device of FIG. 11;

FIGS. 13A and 13B are cross-sectional views of a wire useful with the delivery device of FIG. 11;

FIG. 14 is a simplified cross-sectional view of portions of the delivery device of FIG. 11, including a wire assembled to a stability shaft;

FIG. 15 schematically illustrates portions of the delivery device of FIG. 11, including a handle assembly, stability shaft, and wire;

FIG. 16 is a simplified side view of a prosthetic heart prosthetic heart valve mounted to portions of a delivery device useful as the delivery device of FIG. 11; and

FIG. 17 illustrates use of the delivery device of FIG. 11 in percutaneously delivering a prosthetic heart valve to a target site in accordance with methods of the present disclosure.

### DETAILED DESCRIPTION

Specific embodiments of the present disclosure are now described with reference to the figures, wherein like reference numbers indicate identical or functionally similar elements.

Aspects of the disclosure are beneficial for use with prosthetic heart valves and heart valve repair methods including the implantation of a prosthetic heart valve, particularly, prosthetic heart valves delivered via a transcatheter procedure. As referred to herein, prosthetic heart valves can include a bioprosthetic heart valve structure having tissue leaflets or a synthetic heart valve having polymeric, metallic or tissue-engineered leaflets, and can be specifically configured for replacing or repairing valves of the human heart. In one non-limiting example, the valve of the human heart is an aortic valve, although the systems and methods of the present disclosure can be useful with the mitral, tricuspid, or pulmonary heart valve. The prosthetic heart valves of the present disclosure may be self-expandable, balloon expandable and/or mechanically expandable or combinations thereof. In general terms, the prosthetic heart valves of the present disclosure include a stent or stent frame having an internal lumen maintaining a valve structure (tissue or synthetic), with the stent frame having a normal, expanded condition or arrangement and collapsible to a compressed condition or arrangement for loading within the delivery device. For example, the stents or stent frames are support structures that comprise a number of struts or wire segments arranged relative to each other to provide a desired compressibility and strength to the prosthetic valve. The struts or wire segments are arranged such that they are capable of self-transitioning from, or being forced from, a compressed or collapsed arrangement to a normal, radially expanded arrangement. The struts or wire segments can optionally be formed from a shape memory material, such as a nickel titanium alloy (e.g., nitinol). The stent frame can be laser-cut from a single piece of material, or can be assembled from a number of discrete components.

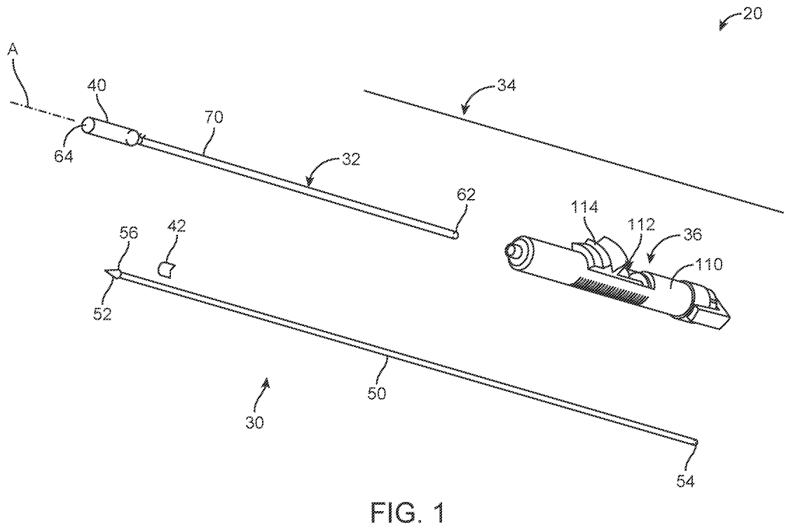

In some embodiments, aspects of the present disclosure relate to delivery device for implanting a prosthetic heart valve at a target site. One example of a transcatheter prosthetic heart valve delivery device 20 in accordance with principles of the present disclosure is shown in FIG. 1. The delivery device 20 includes an inner shaft assembly 30, an outer shaft assembly 32, one or more rods 34, and a handle assembly 36. Details on the various components are provided below. In general terms, however, the delivery device 20 can, in many respects, be any standard construction delivery device, such as, but not limited to, multi-lumen or coaxial construction delivery devices useful for percutaneously delivering and implanting a stented prosthetic heart valve. In some embodiments, the delivery device 20 is configured to provide a loaded or delivery state in which a self-expandable prosthetic heart valve is loaded over the inner shaft assembly 30 in a radially collapsed condition, and retained within a capsule 40 of the outer shaft assembly 32. For example, the inner shaft assembly 30 can include or provide a valve retainer configured to selectively receive a corresponding feature (e.g., posts or eyelets) provided with the prosthetic heart valve stent frame. The outer shaft assembly 32 can be manipulated to proximally withdraw the capsule 40 from over the prosthetic heart valve via operation of the handle assembly 36, permitting the prosthesis to release from the inner shaft assembly 30. In other embodiments, the capsule 40 may not be required, such as when using a balloon expandable prosthetic heart valve, or a self-expandable prosthetic heart valve with other means to retain the prosthetic heart valve in radially collapsed condition. With these and other examples, an optional balloon 42 (schematically illustrated in FIG. 1) can be provided with or carried by the inner shaft assembly 30. In a loaded state, the prosthetic heart valve is crimped over the balloon 42 in a deflated condition; the balloon 42 is subsequently inflated to effect deployment of the prosthetic heart valve. Regardless of the prosthetic heart valve retention and deployment design features incorporated into the delivery device 20, the rod(s) 34 are selectively received and/or advanced within a corresponding slot (not shown) of the outer shaft assembly 32 to increase an ability of the outer shaft assembly 30 to transmit torque (i.e., torqueability) as described below. This enhanced torqueability can be beneficial to a user under many circumstances, for example to arrange the loaded prosthetic heart valve in a desired rotational position relative to native anatomy. The delivery device 20 can optionally include other components that assist or facilitate or control delivery and/or deployment, such as an outer stability tube (not shown).

As a point of reference, various features of the components 30-36 reflected in FIG. 1 and described below can be modified or replaced with differing structures or mechanisms. Thus, the present disclosure is in no way limited to the inner shaft assembly 30, the outer shaft assembly 32, the handle assembly 36, etc., as shown and described below. More generally, then, some delivery devices in accordance with principles of the present disclosure provide features capable of retaining a self-deploying stented prosthetic heart valve (e.g., the capsule 40) and/or capable of deploying a balloon expandable prosthetic heart valve (e.g., the balloon 42), along with one or more components (e.g., the rod(s) 34) capable of increasing a torqueability of the outer shaft assembly 32. In yet other embodiments, the rod(s) 34 and corresponding assembly techniques described below can be useful with any large bore catheter (that is not otherwise carrying a prosthetic heart valve) intend to be advanced around a bend and under circumstances where torqueing the catheter around the bend is desired.

The inner shaft assembly 30 can have various constructions appropriate for supporting a stented prosthetic heart valve. In general terms, the inner shaft assembly 30 is sized and shaped to extend or be co-axially received within a central lumen of the outer shaft assembly 32. The inner shaft assembly 30 includes an inner shaft 50 and a distal tip 52. Depending upon a configuration of the delivery device 20 (e.g., for use with a self-expanding prosthetic heart valve or for use with a balloon expandable prosthetic heart valve), the inner shaft assembly 30 can further include or carry the optional balloon 42 and/or a valve retainer (not shown). The inner shaft 50 extends from a proximal end 54 to a distal end 56. While the inner shaft 50 is illustrated as being an integrally formed body, in other embodiments, two or more shaft members with differing constructions can be separately formed and subsequently assembly to serve as the inner shaft 50. The distal end 56 is secured to the distal tip 52. The distal tip 52 can define a distally tapering outer surface adapted to promote atraumatic contact with bodily tissue. Where provided, the balloon 42 can be secured relative to the inner shaft 50 and/or the outer shaft assembly 32 in various manners as is known in the art, and can be fluidly connected to an inflation source in various manners (e.g., an inflation lumen can be defined in or along the inner shaft 50 that is fluidly connected to an interior of the balloon 42; an inflation passageway can be established between the inner and outer shaft assemblies 30, 32; etc.). The inner shaft assembly 30 can optionally include additional features that may or may not be directly implicated by the view; for example, the inner shaft assembly 30 can form a lumen (e.g., a guidewire lumen) extending from the proximal end 54 to and through the distal tip 52.

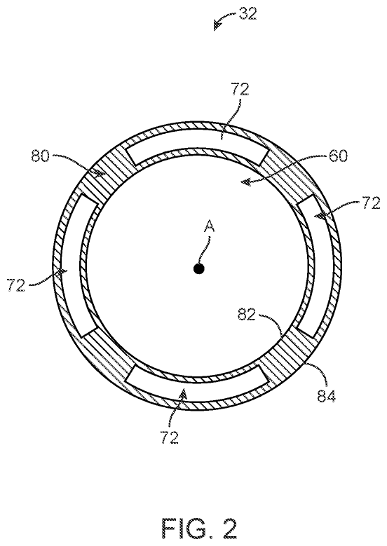

The outer shaft assembly 32 has an elongated shape, defining a longitudinal axis A. The outer shaft assembly 32 defines a central lumen 60 (hidden in FIG. 1, but shown, for example, in FIG. 2) extending from a proximal end 62 to an opposing, distal end 64 (i.e., extending along the longitudinal axis A). In some examples, the outer shaft assembly 32 includes the capsule 40 extending distally from a shaft 70. With these and similar embodiments, the capsule 40 and the shaft 70 can be comprised of differing materials and/or constructions, with the capsule 40 having a longitudinal length approximating (e.g., slightly greater than) a length of the prosthetic heart valve to be used with the delivery device 20. For example, the capsule 40 can have a more stiffened construction as compared to a stiffness of the shaft 70. In other embodiments, the capsule 40 and the shaft 70 can have a more uniform construction. In yet other embodiments, the capsule 40 can be omitted.

Regardless of whether the capsule 40 is provided, the outer shaft assembly 32 defines one or more slots 72 as shown in FIG. 2. As a point of reference, at least a portion of the outer shaft assembly 32 can be formed as or can include a tubular side wall 80. A shape of the side wall 80 defines an interior face 82 opposite an exterior face 84. The central lumen 60 is defined or bounded by the interior face 82. The side wall 80 has a thickness between the interior and exterior faces 82, 84. With these conventions in mind, the slot(s) 72 are defined in and along a thickness of the side wall 80. While FIG. 2 reflects four of the slots 72, any other number, either greater or lesser is also acceptable. With embodiments in each two (or more) of the slots 72 are provided, the slots 72 can be equidistantly spaced from one another about a circumference of the side wall 80. Alternatively, a non-uniform spacing can be provided. Further, where two (or more) of the slots 72 are provided, each of the slots 72 can have an identical construction as shown; alternatively, the slots 72 can vary from one another in terms of size and/or shape. Regardless, the slot(s) 72 are configured to slidably receive a corresponding one of the rods 34 (FIG. 1) as described in greater detail below.

With reference between FIGS. 1 and 2, regardless of the number provided, each of the slots 72 extends to and is open at the proximal end 62 of the outer shaft assembly 32 in some embodiments. Alternatively, one or more of the slots 72 can terminate distal the proximal end 62, but extends through and is open at the exterior face 84 (e.g., at a port for receiving one of the rods 34). In some embodiments, each of the slots 72 terminate proximal the distal end 64 (or, with embodiments in which the outer shaft assembly 32 includes the capsule 40, the slots 72 can terminate proximal the capsule 40). Alternatively, one or more of the slots 72 can extend to and be open at the distal end 64. In some embodiments, and for reasons made clear below, in some embodiments, the slot(s) 72 extend distally from a location proximate or at the proximal end 62 to location at least 75% of a length of the outer shaft assembly 32.

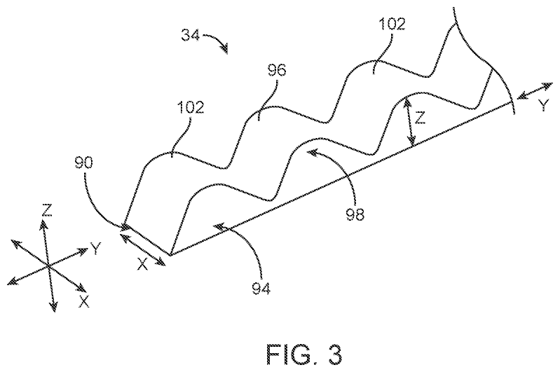

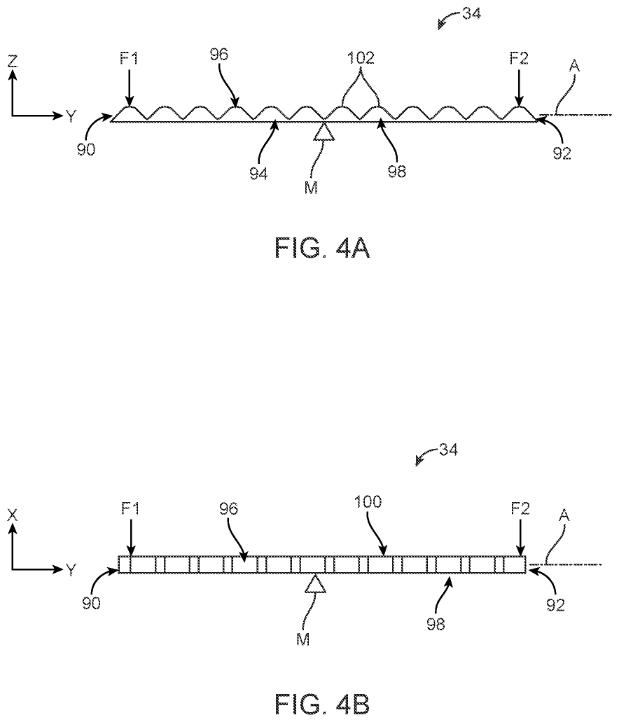

One embodiment of the rod 34 is shown in FIGS. 3, 4A and 4B. In general terms, the rod 34 has an elongated shape, and is sized to be slidably received within a corresponding one of the slots 72 (FIG. 2). Further, the rod 34 is formed of a relatively stiff and resilient material (e.g., metal, plastic, fiber composite, etc.) and is configured to exhibit a flexibility characteristic or property in a first plane or direction that is substantively different from a flexibility in a second plane orthogonal to the first plane. As a point of reference, in the perspective view of FIG. 3, directional axes X, Y and Z are identified; the X axis corresponds with a width dimension or direction of the rod 34; the Y axis is perpendicular to the Y axis and corresponds with a length dimension or direction of the rod 34; the Z axis is perpendicular to the X and Y axes, and corresponds with a thickness or height dimension or direction of the rod 34. FIG. 4A is a side view of the rod 34, corresponding with a plane defined by the Y, Z axes; FIG. 4B is a top view of the rod, corresponding with a plane defined by the X, Y axes. The plane of the view of FIG. 4A is orthogonal or transverse to the plane of the view of the FIG. 4B. With these conventions in mind, a flexibility of the rod 34 in the plane of FIG. 4A is greater than a flexibility of the rod 34 in the plane of FIG. 4B.

Alternatively stated, the rod 34 defines a first end 90 opposite a second end 92, a lower surface 94 (referenced generally in FIG. 3 and hidden in FIG. 4B) opposite an upper surface 96, and a first side surface 98 opposite a second side surface 100 (hidden in FIGS. 3 and 4A). The width of the rod 34 is defined as the distance between the first and second side surfaces 98, 100 (i.e., dimension along the X axis). In some embodiments, the width (i.e., dimension along the X axis) of the rod 34 is substantially uniform (i.e., within 5% of a truly uniform width) along the length (i.e., from the first end 90 to the second end 92). Further, in some embodiments, a major plane defined by each of the first and second side surfaces 98, 100 is substantially perpendicular (i.e., within 5% of a truly perpendicular relationship) with a major plane defined by the lower surface 94. Finally, the elongated shape of the rod 34 defines a central axis A. With these designations in mind, the orthogonal plane flexibility characteristics can be described by a flexibility of the rod 34 in a first plane perpendicular to the central axis A and intersecting the lower and upper surfaces 94, 96 (e.g., the plane of FIG. 4A, a plane defined by the Y and Z axes) being greater than a flexibility of the rod 34 in second plane that is orthogonal to the first plane and that intersects the first and second side surfaces (e.g., the plane of FIG. 4B, a plane defined by the X and Y axes).

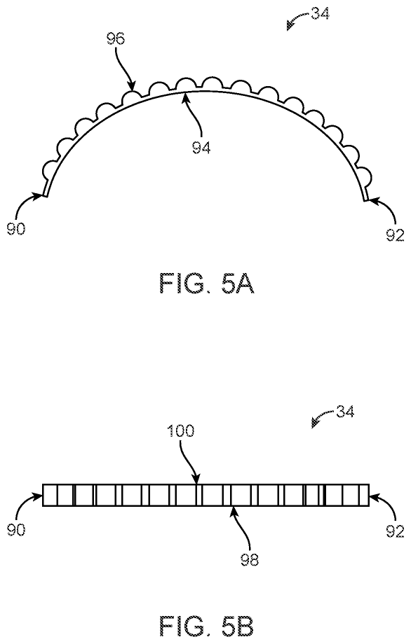

Flexibility characteristics of the rod 34 can further be described with reference to loads or forces applied to the rod 34. For example, where identical point forces or loads F1, F2 are applied to the first and second ends 90, 92, respectively, in the plane of FIG. 4A and a mid-point M is fixed point (i.e., the forces F1, F2 are applied to upper surface 96 with a fulcrum on the lower surface 94 at the mid-point M), the rod 34 will readily flex; where the same forces F1, F2 are applied to the first and second ends 90, 92 in the plane of FIG. 4B (i.e., the forces F1, F2 are applied to the second side surface 100 with a fulcrum on the first side surface 98 at the mid-point M), the rod 34 will not readily flex. Alternatively stated, the rod 34 is configured such that the rod 34 overtly resists, and does not deform or flex, when the forces F1, F2 are applied to the ends 90, 92 in the plane of FIG. 4B, but does not overtly resist, and will deform or flex, when the same forces F1, F2 are applied in the plane of FIG. 4A. Thus, FIG. 5A represents an effect of the forces F1, F2 as applied to the upper surface 96 and a fulcrum is at the mid-point M along the lower surface 94 (i.e., in the plane of FIG. 4A); the rod 34 deforms or flexes (i.e., the rod 34 transitions from the arrangement of FIG. 4A to the arrangement of FIG. 5A). FIG. 5B represents the effect of the same forces F1, F2 when applied to the second side surface 100 and a fulcrum is at the mid-point M along the first side surface 98 (i.e., in the plane of FIG. 4B); the rod 34 does not deform or flex (i.e., an arrangement of the rod 34 in FIGS. 4B and 5B is essentially the same).

The rods of the present disclosure can assume various forms or formats that exhibit the orthogonal plane flexibility characteristics described above. For example, in the non-limiting embodiment of FIGS. 4A and 4B, the rod 34 can be shaped to define a varying thickness (i.e., dimension along the Z axis) along a length thereof. Alternatively stated, a thickness of the rod 34 is defined between the lower and upper surfaces 94, 96; the thickness varies along the length of the rod 34 (i.e., from the first end 90 to the second end 92). In some embodiments, the varying thickness attribute can be described as the rod 34 having a corrugated shape along the length thereof. In some embodiments, the varying thickness attribute can be described as the lower surface 94 being substantially flat or planar (i.e., within 5% of a truly flat or planar surface), whereas the upper surface 96 has a wavy-like shape. In some embodiments, the varying thickness attribute can be described as the upper surface 96 defining a series of protrusions 102 relative to the lower surface 94. Other shapes or constructions can also be employed.

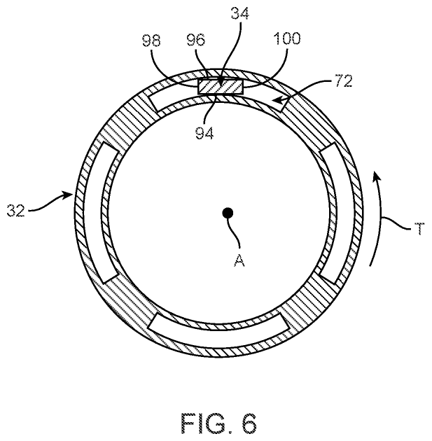

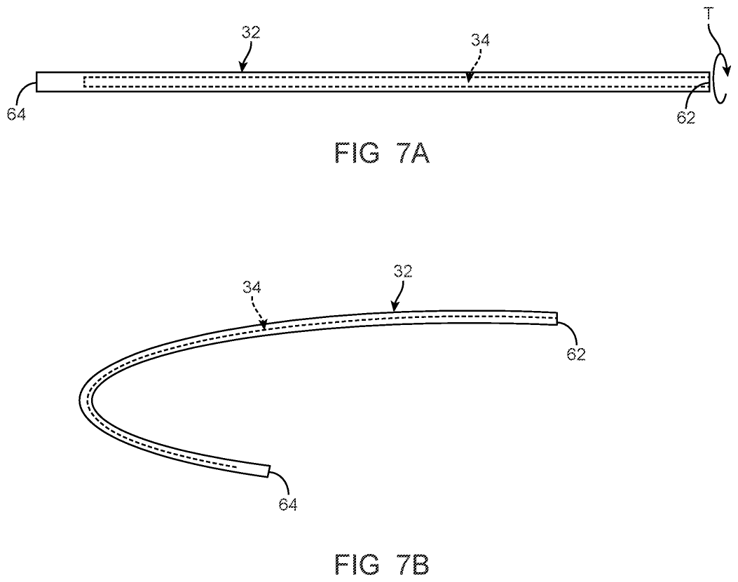

Regardless of an exact construction, the rod 34 is sized and shaped to be slidably received in one of the slots 72 of the outer shaft assembly 32 as shown in FIG. 6. In some embodiments, the rod 34 is or can be arranged within the slot 72 such that the upper surface 96 is radially opposite the lower surface 94 relative to the longitudinal axis A. Alternatively stated, the rod 34 is or can be arranged such that the lower surface 94 is closer to the longitudinal axis A as compared to the upper surface 96; the opposing side surfaces 98, 100 are circumferentially aligned relative to the longitudinal axis A. With this construction, and commensurate with the descriptions above, the rod 34 serves to enhance a torqueability of the outer shaft assembly 32, overtly reinforcing the outer shaft assembly 32 relative to, or off-setting, an applied rotational force or torque. In this regard, it will be recalled that the rod 34 is configured to have minimal flexibility in the plane of FIG. 4B. That is to say, the rod 34 does not easily deform, flex, or deflect in response to rotational or torque-type forces applied to the opposing side surfaces 98, 100. Relative to the arrangement of FIG. 6, then, the rod 34 resists or “stiffens” the outer shaft assembly 32 relative to a torque T applied onto the outer shaft assembly. Thus, and with additional reference to the simplified representation of FIG. 7A, a rotational force or torque T applied to the outer shaft assembly 32 near or proximate the proximal end 62 is readily transferred to or near the distal end 64 due to the presence of the rod 34. Thus, rotation of the proximal end 62 results in a substantially similar rotation of the distal end 64. Were the rod 34 not present, the torque T applied at the proximal end 62 would not readily transfer to or near the distal end 32 (e.g., the outer shaft assembly 32 may twist or deform along a length thereof). However, the flexible nature of the rod 34 in an orthogonal plane (i.e., the plane of FIG. 4A) allows the rod 34 to readily conform to bends or curves in the outer shaft assembly 32 during insertion/advancement. Thus, for example, under circumstances where the outer shaft assembly 32 is forced to the assume a non-linear shape in extension from the proximal end 62 to the distal end 64 as shown in FIG. 7B, as the rod 34 is advanced (within the slot 72 (FIG. 6)) distally toward the distal end 64, the rod 34 readily flexes and “tracks” to the shape or contour of the outer shaft assembly 32.

Returning to FIG. 1, the handle assembly 36 can assume various forms appropriate for user handling and operation of the delivery device 20. In some embodiments, the handle assembly 36 includes a housing 110 and one or more actuator mechanisms 112 (referenced generally). The housing 110 generally provides a surface for convenient handling and grasping by a user, and may have the generally cylindrical shape as shown, although other shapes and sizes are also acceptable. The housing 110 maintains the actuator mechanism 112, with the handle assembly 36 configured to facilitate sliding movement of the outer shaft assembly 32 relative to the inner shaft assembly 30 (and/or vice-versa). In one simplified construction of the actuator mechanism 112, a user interface or actuator 114 is slidably retained by the housing 110, and is coupled to the proximal end 62 of the outer shaft assembly 32. The proximal end 54 of the inner shaft assembly 30 is secured to the housing 110. With this optional construction, sliding movement of the actuator 114 co-axially advances/retracts the outer shaft assembly 32 relative to the inner shaft assembly 30. Although shown as a slide mechanism, other constructions and/or devices may be used to retrace/advance the outer shaft assembly 32 relative to the inner shaft assembly 30 (and/or vice-versa), such as, but not limited to, rotating mechanisms, sliding mechanisms that are coaxially disposed over the inner shaft assembly 30, combinations of rotating and sliding mechanisms, and other advancement/retraction mechanisms apparent to those of ordinary skill in the art. The handle assembly 36 can optionally include one or more additional components or mechanism; for example with embodiments in which the inner shaft assembly 30 includes or carries the balloon 42, the handle assembly 36 can optionally include or carry features that facilitate delivery of an inflation medium to the balloon 42. In other embodiments, the handle assembly 36 does not include an actuator for moving the outer shaft assembly 32 relative to the inner shaft assembly 30 (or vice-versa), such as with embodiments in which the capsule 40 is omitted.

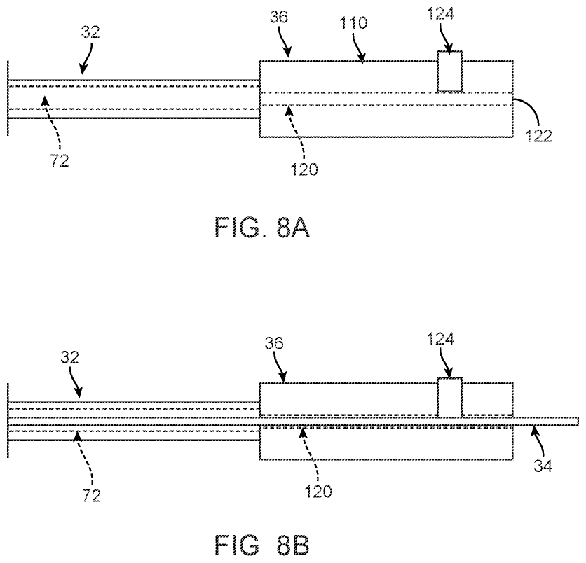

In some embodiments, the handle assembly 36 can include or carry one or more mechanisms or features that facilitate insertion and/or advancement and retraction of the rod(s) 34 relative to the outer shaft assembly 32 (and in particular the corresponding slot 72 (FIG. 2)). For example, FIG. 8A is a simplified representation of the outer shaft assembly 32 assembled to, and extending distally from, the handle assembly 36. The housing 110 forms or defines a passage 120 that, upon final assembly, is aligned with one of the slots 72 in the outer shaft assembly 32 (it being understood that where the outer shaft assembly 32 provides two or more of the slots 72, the housing 110 will define a corresponding number of the passages 120, each aligned with a respective one of the slots 72). The passage 120 is sized to slidably receive one of the rods 34 (FIG. 1), and is open to an exterior of the housing 110 (e.g., opening 122). In some embodiments, the handle assembly 36 can include a locking mechanism 124 that is generally configured to selectively engage the rod 34 as described in greater detail below.

With the above construction, the rod 34 (FIG. 1) can be inserted into the slot 72 of the outer shaft assembly 32 via the passage 120. That is to say, in some embodiments, the delivery device 20 (FIG. 1) is provided to an end user with the rod(s) 34 separate from or not otherwise carried by the handle assembly 36 (or the outer shaft assembly 32). With these and related embodiments, where use of the rod 34 is desired (e.g., to reinforce the outer shaft assembly 32), the user simply inserts the rod 34 into the opening 122 and advances the rod 34 distally through the passage 120 and into the slot 72 as generally reflected by FIG. 8B. In other embodiments, the delivery device 20 is provided to an end user with the rod(s) 34 disposed within the corresponding passage 120 and advanced only a small distance, if at all, into the corresponding slot 72. Where reinforcement of the outer shaft assembly 32 is desired, the user then distally advances the rod 34 along the corresponding slot 72. Regardless, the optional locking mechanism 124 can be operated by a user to secure or lock the rod 34 relative to the housing 110, and thus relative to the outer shaft assembly 32, at a desired longitudinal arrangement, for example by exerting a locking force onto the rod 34 along the passage 120 as generally reflected by FIG. 8B. The locking mechanism 124 can assume various forms (e.g., a spring-loaded actuator, a rotating mechanism, etc.), and in other embodiments can be omitted. With these and other embodiments, when reinforcement of the outer shaft assembly 32 is no longer necessary or not otherwise desired by the user, the rod 34 can be proximally retracted along the slot 72 and the passage 120.

Returning to FIG. 1, the transcatheter prosthetic heart valve delivery device 20 is useful for percutaneously delivering and deploying a prosthetic heart valve to any of the four native heart valves. In general terms, methods of the present disclosure are the same as or similar to techniques conventionally employed whereby a prosthetic heart valve is loaded to the delivery device 20, manipulated through a vasculature of the patient to target site, and then deployed at the target site. In some examples, the delivery device 20 is beneficially employed with procedures in which rotation of the loaded prosthetic heart valve at or near the target site is desired as described below.

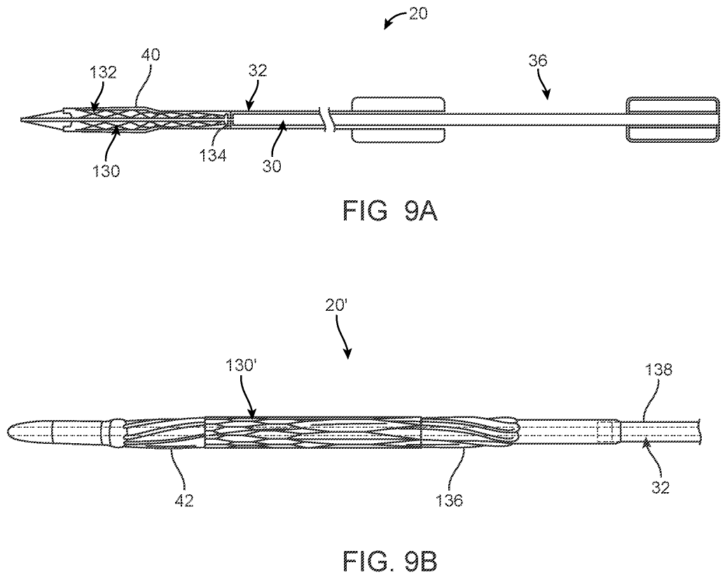

For example, FIG. 9A reflects, in simplified form, one example of the delivery device 20 loaded with a prosthetic heart valve 130 in a compressed state. With the example of FIG. 9A, the prosthetic heart valve 130 includes a self-expanding stent 132 that is crimped over the inner shaft assembly 30 and secured to an optional valve retainer 134 provided with inner shaft assembly 30. The outer shaft assembly 32 includes the optional capsule 40 that is located over the prosthetic heart valve 130, maintaining the prosthetic heart valve 130 in the compressed state. The handle assembly 36 is schematically shown, and generally reflects that the outer shaft assembly 32 can be proximally retracted relative to the inner shaft assembly 30 (and thus relative to the prosthetic heart valve 130). As mentioned above, in other embodiments, the prosthetic heart valve 130 can instead include a balloon expandable stent; with these and related embodiments, the capsule 40 may or may not be provided, and prosthetic heart valve 130 can be compressed over a balloon (not shown) as loaded to the delivery device 20. For example, FIG. 9B is a representation of the delivery device 20′ formatted for use with a balloon expandable prosthetic heart valve 130′. The delivery device 20′ includes the balloon 42. A proximal region 136 of the balloon 42 overlies a distal region 138 of the outer shaft assembly 32, and the prosthetic heart valve 130′ is compressed or crimped onto the deflated balloon 42. As a point of reference, while the outer shaft assembly 32 is shown as being tapered or stepped to a smaller diameter to accommodate the balloon 42, in other embodiments a thickness or outer diameter of the outer shaft assembly 32 along the distal region 138 can be increased to house the rod(s) 34 (FIG. 1). Regardless of an exact format of the delivery device 20, 20′, in the initial loaded condition of FIGS. 9A and 9B, the rod(s) 34 (FIG. 1) have not been inserted or distally advanced within the outer shaft assembly 32.

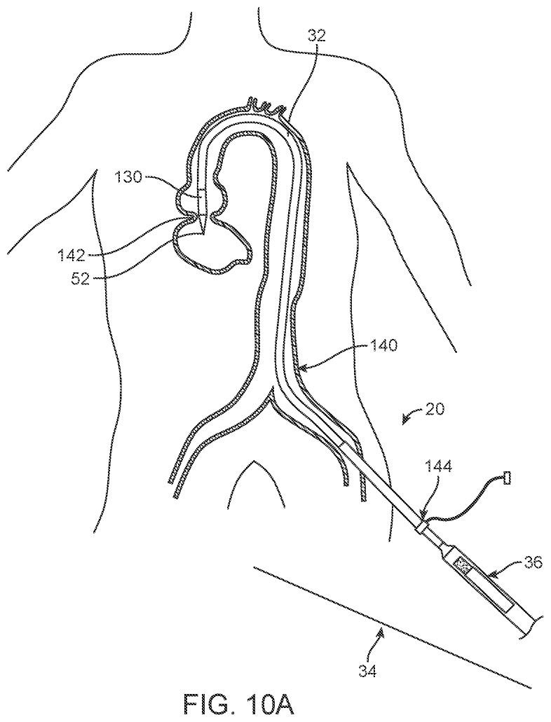

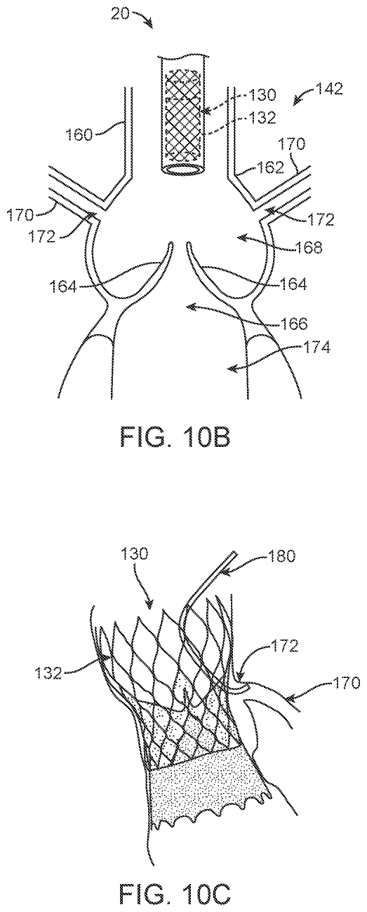

The loaded delivery device 20 is then manipulated to percutaneously deliver the prosthetic heart valve 130 (in the compressed state) through a vasculature of the patient to a target site. For example, FIG. 10A illustrates the loaded delivery device 20 having been advanced through a patient's vasculature 140 to deliver the prosthetic heart valve 130 (hidden, but referenced generally in FIG. 10A) to an aortic valve target site 142 (e.g., at the stage of delivery of FIG. 10A, the tip 52 is proximate the aortic valve target site 142). In some embodiments, an introducer 144 can be used to assist in establishing a portal to a bodily lumen (e.g., femoral artery) of the patient. Regardless, the user manipulates the handle assembly 36 to direct the compressed prosthetic heart valve 130 through or along the vasculature 140. In many instances, the vasculature 140 encountered by the delivery device 20 can be highly tortuous. For example, in accessing the aortic valve target site 142, the compressed prosthetic heart valve 130 must track along or traverse the aortic arch. During this tracking stage of the delivery process, then, the rod(s) 34 are removed from, or not otherwise advanced through, the outer shaft assembly 32 (as generally shown). In this state, the delivery device 20 readily passes through or tracks along the vasculature 140.

In the delivery stage of FIG. 10A, the prosthetic heart valve 130 is proximate the target site 142 and is still secured to the delivery device 20. Prior to complete deployment from the delivery device 20 at the target site 142, the user may desire to spatially rotate the prosthetic heart valve 130. For example, the user may evaluate a rotational arrangement of the prosthetic heart valve 130 relative to native anatomy of the target site 142 and decide that a different rotational arrangement is preferred. Under these and other circumstances, the rod(s) 34 are then advanced within the outer shaft assembly 32 as described above, increasing an overall stiffness of the outer shaft assembly 32. In this regard, the rod(s) 34 can readily track along the curves or bends formed in the outer shaft assembly 32. With the rod(s) 34 in place, a torque or rotational force applied by the user onto the handle assembly 36 is transferred to the distal region of the outer shaft assembly 32, thereby rotating the prosthetic heart valve 130. In some embodiments, a nearly 1:1 response between rotation of the handle assembly 36 and the prosthetic heart valve 130 can be provided with the delivery device 20 located in the patient's anatomy. Once a desired rotational arrangement of the prosthetic heart valve 130 has been achieved, the rod(s) 34 can be retracted or removed, followed by operation of the delivery device 20 to deploy the prosthetic heart valve 130 at the target site 142.

A desire or need to adjust a rotational arrangement of the prosthetic heart valve 130 relative to the target site 142 can arise under various circumstances. For example, FIG. 10B illustrates, in simplified form, the compressed prosthetic heart valve 130 loaded within the delivery device 20 and located proximate the aortic valve target site 142. Generally, patient anatomy at and adjacent the aortic valve 142 includes an aorta 160, sinotubular junction (“STJ”) 162, native valve leaflets 164, aortic valve annulus 166, sinus region 168, coronary arteries (or “coronaries”) 170 each having a coronary ostium 172, and left ventricle 174. In some instances, a size of the prosthetic heart valve 130 and/or the patient's anatomy (e.g., short and/or narrow sinuses) may result in a structure of the prosthetic heart valve 130 being proximate the coronary ostia 172 upon final implant, for examples struts of the stent frame 132. The potential for obstruction of the coronary ostia 172 is further heightened when the prosthetic heart valve 130 is being deployed or implanted within a previously implanted prosthesis. Thus, a user may desire to rotationally arrange the prosthetic heart valve 130 relative to the native anatomy so as to minimize obstructions to the coronary ostia 172. For example, and as shown in FIG. 10C, a user may desire to rotate the prosthetic heart valve 130 relative to the native anatomy so as to align openings in the stent 132 with the coronary ostia 172; when so-aligned, an interventional device 180 (e.g., access catheter) can more easily access the coronary arteries 170 as part of a subsequent procedure. The delivery devices 20 (FIG. 1) of the present disclosure facilitate desired rotation of the prosthetic heart valve 130 to meet these, and many other, needs.

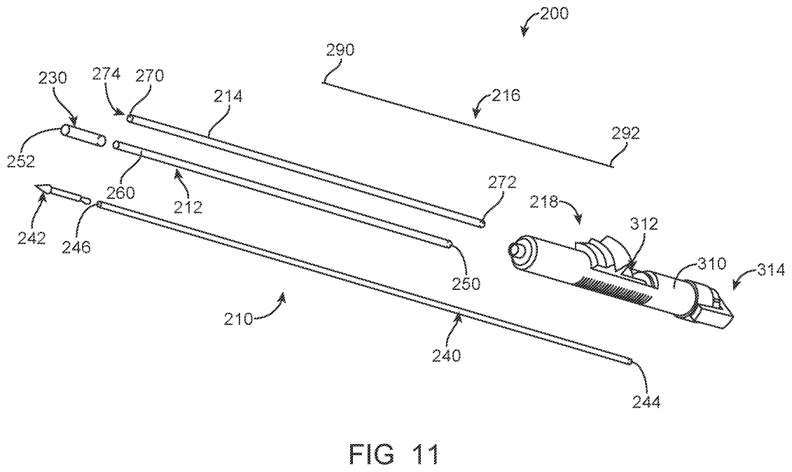

Another embodiment of a transcatheter prosthetic heart valve delivery device 200 in accordance with principles of the present disclosure is shown in FIG. 11. The delivery device 200 includes an inner shaft assembly 210, an outer shaft assembly 212, a stability shaft 214, at least one wire 216, and a handle assembly 218. Details on the various components are provided below. In general terms, however, the delivery device 200 can, in many respects, be any standard construction delivery device, such as, but not limited to, multi-lumen or coaxial construction delivery devices useful for percutaneously delivering and implanting a stented prosthetic heart valve. In some embodiments, the delivery device 200 is configured to provide a loaded or delivery state in which a self-expandable prosthetic heart valve (not shown) is loaded over the inner shaft assembly 210 in a radially collapsed condition, and compressively retained within a capsule 230 of the outer shaft assembly 212. The outer shaft assembly 212 can be manipulated to proximally withdraw the capsule 230 from over the prosthetic heart valve via operation of the handle assembly 218, permitting the prosthesis to release from the inner shaft assembly 210. The stability shaft 214 is received over the outer shaft assembly 212, and serves to frictionally isolate the outer shaft assembly 212 from a separate introducer device (not shown) as part of a transcatheter prosthetic heart valve delivery and deployment procedure. As is understood by one of ordinary skill, the stability shaft 214 acts as a distance stabilizing member to help set and maintain the distance between the handle assembly 218 and the introducer positioned in the patient's artery to thereby promote accurate deployment of the prosthetic heart valve at a target site. The stability shaft 214 also reduces the likelihood of unexpected movements of the capsule 230 at the target site. With this in mind, the wire 216 is affixed to the stability shaft 214 and extends to the handle assembly 218. The handle assembly 218 can be operated by a user to selectively engage or lock the wire 216, with a resulting tension in the wire 216 creating or generating an enhanced bending stiffness in the stability shaft 214. The elevated bending stiffness can be desirable or beneficial in many scenarios, for example to overcome forces encountered when retracting the capsule to deploy the prosthetic heart valve.

As a point of reference, various features of the components 210-218 reflected in FIG. 11 and described below can be modified or replaced with differing structures or mechanisms. Thus, the present disclosure is in no way limited to the inner shaft assembly 210, the outer shaft assembly 212, the handle assembly 218, etc., as shown and described below. More generally, then, some delivery devices in accordance with principles of the present disclosure provide features capable of retaining a self-deploying stented prosthetic heart valve (e.g., the capsule 230), along with one or more components (e.g., the wire(s) 216) capable of selectively increasing a bending stiffness of the stability shaft 214.

The inner shaft assembly 210 can have various constructions appropriate for supporting a stented prosthetic heart valve. In general terms, the inner shaft assembly 210 is sized and shaped to extend within a central lumen of the outer shaft assembly 212. The inner shaft assembly 210 includes an inner shaft 240 and a distal tip 242. The inner shaft 240 extends from a proximal end 244 to a distal end 246. While the inner shaft 240 is illustrated as being an integrally formed body, in other embodiments, two or more shaft members with differing constructions can be separately formed and subsequently assembly to serve as the inner shaft 240. The distal end 246 is secured to the distal tip 242. The distal tip 242 can define a distally tapering outer surface adapted to promote atraumatic contact with bodily tissue. The inner shaft assembly 210 can optionally include additional features that may or may not be directly implicated by the view; for example, the inner shaft assembly 210 can form a lumen (e.g., a guidewire lumen) extending from the proximal end 244 to and through the distal tip 242.

The outer shaft assembly 212 defines a central lumen (hidden) extending from a proximal end 250 to an opposing, distal end 252. The outer shaft assembly 212 includes the capsule 230 extending distally from a shaft 260. With these and similar embodiments, the capsule 230 and the shaft 260 can be comprised of differing materials and/or constructions, with the capsule 230 having a longitudinal length approximating (e.g., slightly greater than) a length of the prosthetic heart valve (not shown) to be used with the delivery device 200. For example, the capsule 230 can have a more stiffened construction as compared to a stiffness of the shaft 260. In other embodiments, the capsule 230 and the shaft 260 can have a more uniform construction.

The stability shaft 214 can generally have any construction conventionally employed. The stability shaft 214 extends from a distal end 270 to a proximal end 272 and forms a central lumen 274 (referenced generally) sized to be slidably received over the outer shaft assembly 212. In some embodiments, the stability shaft 214 can be formed of a polymeric material with an associated reinforcement layer.

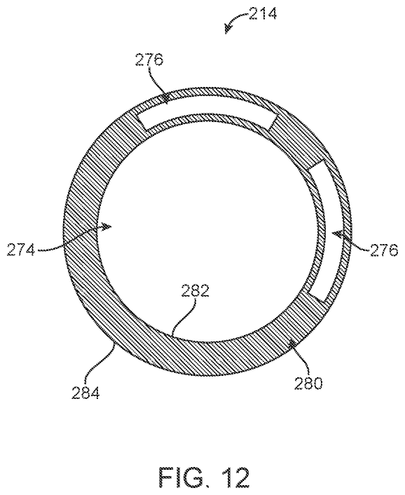

As described below, the wire(s) 216 can be secured to the stability shaft 214 in various manners. In some embodiments, the stability shaft 214 incorporates one or more features that promote connection with the wire(s) 216. For example, and as shown in FIG. 12, the stability shaft 214 can form or define one or more slots 276 in addition to the central lumen 274. As a point of reference, at least a portion of the stability shaft 214 can be formed as or can include a tubular side wall 280. A shape of the side wall 280 defines an interior face 282 opposite an exterior face 284. The central lumen 274 is defined or bounded by the interior face 282. The side wall 280 has a thickness between the interior and exterior faces 282, 284. With these conventions in mind, the slot(s) 276 are defined in and along a thickness of the side wall 280. The number of slots 276 corresponds with the number of wires 216 (FIG. 11). Thus, while FIG. 12 reflects two of the slots 276, any other number, either greater or lesser is also acceptable. With embodiments providing two of the slots 276, the slots 276 can be arranged at approximately 90 degrees from one another (relative to a circumference of the stability shaft 214) for reasons made clear below. Alternatively, other arrangements can be provided. Regardless, the slot(s) 276 are configured to receive a corresponding one of the wires 216 as described in greater detail below.

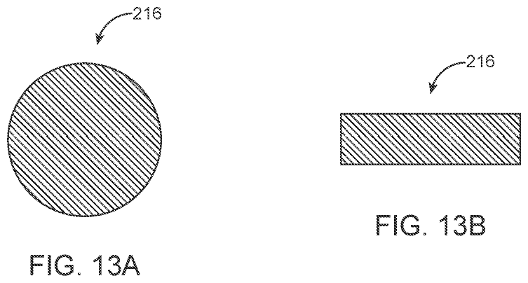

Returning to FIG. 11, the wire 216 can assume various forms appropriate for supporting or reinforcing the stability shaft 214. In some embodiments, the wire 216 is formed of metal (e.g., stainless steel), hardened plastic, composite material, etc. The wire 216 terminates at opposing, leading and trailing ends 290, 292. The wire 216 can have various cross-sectional shapes, for example circular (FIG. 13A), flat or rectangular (FIG. 13B), irregular, etc. The cross-sectional shape can be substantially uniform along the wire 216 in extension between the ends 290, 292; in other embodiments, the wire 216 can have a varying cross-sectional shape (e.g., one or more segments of the wire 216 can have a flattened cross-sectional shape, whereas other segment(s) have a rounded or circular cross-sectional shape).

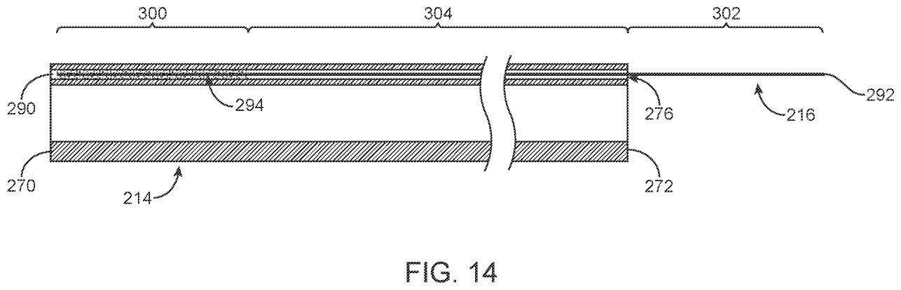

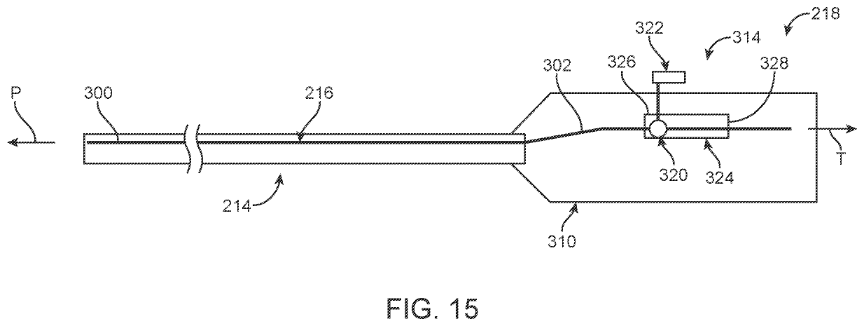

FIG. 14 provides a simplified illustration of the stability shaft 214 and one of the wires 216 upon final assembly. The wire 216 is located with the slot 276, with the leading end 290 located at or in close proximity (e.g., within 5 centimeters) to the distal end 270. In some embodiments, a length of the wire 216 (i.e., linear distance from the leading end 290 to the trailing end 292) is greater than a length of the stability shaft 214 (i.e., linear distance from the distal end 270 to the proximal end 272). With this configuration, the trailing end 292 of the wire 216 is located away from, or proximally beyond, the proximal end 272 of the stability shaft 214 for reasons made clear below.

While the wire 216 is disposed within the slot 276, only a portion of the wire 216 is directly affixed to the stability shaft 214. For example, a material bond 294 is shown in FIG. 14 between a surfaces of the stability shaft 214 and the wire 216 near or at the leading end 290. The material bond 294 can assume various forms appropriate for fixing that section of the stability shaft 214, otherwise in contact with the bond 294, with the stability shaft 276 (e.g., the material body 294 can be an adhesive, lamination of material of the stability shaft 276 to a surface of the wire 216, mechanical or ultrasonic bond, an attachment body, etc.).

From the above descriptions, in the assembled state of FIG. 14, the wire 216 can be viewed or designated as defining a leading section 300, a trailing section 302 opposite the leading section 300, and an intermediate section 304 between the trailing and leading sections 300, 302. The leading section 300 includes the leading end 290 and is directly affixed to the stability shaft 214. The trailing section 302 includes the trailing end 292 and extends proximally beyond the proximal end 272 of the stability shaft 214. The intermediate section 304 extends from the leading section 300 through the slot 276 to the proximal end 272, and is free of direct, physical fixation to the stability shaft 214. Thus, apart from direct fixation at the leading section 300, the stability shaft 214 can deform or deflect or slide relative to the intermediate section 304 of the wire 216 (so long as the trailing section 302 is not otherwise spatially fixed relative to the proximal end 272). In some embodiments, a length of the intermediate section 304 is greater than a length of the leading section 300; for example, the intermediate section 304 is at least 1.5× longer than the leading section 300, alternatively at least 2× longer, alternatively at least 3× longer.

While FIG. 14 reflects an optional construction in which the stability shaft 214 forms the slot 276 for receive the wire 216, other configurations are also acceptable that may or may not include the wire 216 being located within a thickness of the stability shaft 214. For example, in other embodiments, the wire 216 can be located along an exterior of the stability shaft 214 with only a small section (e.g., akin to the leading section 300) directly, physically affixed to the stability shaft 214. With these and related embodiments, an intermediate section of the wire 216 can be loosely connected to the stability shaft 214 and a trailing section extends proximally beyond the proximal end 272.

Returning to FIG. 11, the handle assembly 218 can assume various forms appropriate for user handling and operation of the delivery device 200. In some embodiments, the handle assembly 216 includes a housing 310, an outer shaft actuator mechanism 312 (referenced generally), and a locking mechanism 314 (referenced generally). The housing 310 generally provides a surface for convenient handling and grasping by a user, and may have the generally cylindrical shape as shown, although other shapes and sizes are also acceptable. The housing 310 maintains the outer shaft actuator mechanism 312, with the handle assembly 218 configured to facilitate sliding movement of the outer shaft assembly 212 relative to the inner shaft assembly 210 (and/or vice-versa). In one simplified construction of the outer shaft actuator mechanism 312, a user interface or actuator 320 is slidably retained by the housing 310, and is coupled to the proximal end 250 of the outer shaft assembly 212. The proximal end 244 of the inner shaft assembly 210 is secured to the housing 310. With this optional construction, sliding movement of the actuator 320 co-axially advances/retracts the outer shaft assembly 212 relative to the inner shaft assembly 210. Although shown as a slide mechanism, other constructions and/or devices may be used to retrace/advance the outer shaft assembly 212 relative to the inner shaft assembly 210 (and/or vice-versa), such as, but not limited to, rotating mechanisms, sliding mechanisms that are coaxially disposed over the inner shaft assembly 210, combinations of rotating and sliding mechanisms, and other advancement/retraction mechanisms apparent to those of ordinary skill in the art.

The locking mechanism 314 is carried by the housing 310, and can assume various forms. In general terms, the locking mechanism is configured to interface with the wire 216, providing a user with the ability to selectively lock the wire(s) 216 relative to the housing 310, and the optional ability to tension the wire(s) 216. Where the delivery device 200 includes two (or more) of the wires 216, the locking mechanism 314 can be configured to simultaneously interface with all of the wires 216. Alternatively, multiple locking mechanisms 314 can be provided, one for each of the wires 216.

One example of the locking mechanism 314 is shown is simplified form in FIG. 15, along with the stability shaft 214 and the wire 216 as connected with the housing 310. Commensurate with the explanations above, the trailing section 302 of the wire 216 extends proximally from the stability shaft 214 into an interior of the housing 310. The locking mechanism 314 includes a lock unit 320 (referenced generally) and an actuator 322. The lock unit 320 is slidably disposed within a channel 324 formed or carried by the housing 310, and is configured to selectively engage the trailing section 302 of the wire 216. For example, housing 310 can include or carry surface features that route the trailing section 302 to the channel 324 for passage through the lock unit 320. The lock unit 320 is operable to provide a locked state and an unlocked state. In the locked state, the wire 216 is rigidly grasped or clamped by the lock unit 320, and cannot slide relative to the lock unit 320 (e.g., the lock unit 320 can include a clamping device, toothed surface(s), etc., that, when directed into engagement with the wire 216, robustly engages the wire 216). In some embodiments, a cross-sectional shape of the wire 216 along at least the trailing section 302 can be relatively flat or rectangular to be more easily grasped. Regardless, in the unlocked state, the wire 216 can freely slide relative to the lock unit 320. The actuator 322 is operable by a user to switch or actuate the lock unit 320 between the locked and unlocked states. Further, the actuator 322 is slidably maintained relative to the housing 310 and can be manipulated by a user to slide or otherwise move the lock unit 320 along the channel 324 between opposing, leading and trailing sides 326, 328.

With this one example embodiment, then, in the unlocked state, because the trailing section 302 can freely slide relative to the handle assembly 218, where a pulling force P is exerted on the wire 216, tension is not generated in the wire 216. For example, recalling that the leading section 300 of the wire 216 is affixed to the stability shaft 214, as the stability shaft 214 is caused to bend or articulate (such as when traversing a patient's vasculature), the pulling force P is exerted on the leading section 300. Under these circumstances, a remainder of the wire 216 is allowed to freely slide relative to the handle assembly 218 and is not brought into tension. Thus, in the unlocked state, the wire 216 generates minimal, if any, additional stiffness in the stability shaft 214. In the locked state, the locked state, the lock unit 320 is clamped onto the trailing section 302. When the pulling force P is applied to the leading section 300, the wire 216 is caused to slide distally. The lock unit 320 moves with the wire 216, sliding along the channel 324 until brought into contact with the leading side 326. An interface between the lock unit 320 and the leading side 326 prevents further movement of the lock unit 320, and thus of the trailing section 302. As a result, the applied pulling force P generates tension in the wire 216, with this tension effectively increasing a bending stiffness of the stability shaft 214. Further, while in the locked state, a user can generate or increase tension in the wire 216 by manipulating the actuator 322 to slide the lock unit 320 (and thus the trailing section 302) in a direction of the trailing side 328 of the channel 324. Because the leading section 300 is affixed to the stability shaft 214, this force movement exerts a tension force T onto the wire 206.

The locking mechanism 314 can assume other forms that may or may not include lock unit 320 and/or actuator 322 as shown and described. Any device or mechanism appropriate for selectively locking the wire 216 relative the handle assembly 218 in a manner that maintains tension in the wire 216 in a locked state and does not maintain tension in an unlocked state is acceptable.

Returning to FIG. 11, the transcatheter prosthetic heart valve delivery device 200 is useful for percutaneously delivering and deploying a prosthetic heart valve to any of the four native heart valves. In general terms, methods of the present disclosure are the same as or similar to techniques conventionally employed whereby a prosthetic heart valve is loaded to the delivery device 200, manipulated through a vasculature of the patient to target site, and then deployed at a the target site. In some examples, the delivery device 20 is beneficially employed with procedures in which rotation of the loaded prosthetic heart valve at or near the target site is desired as described below.

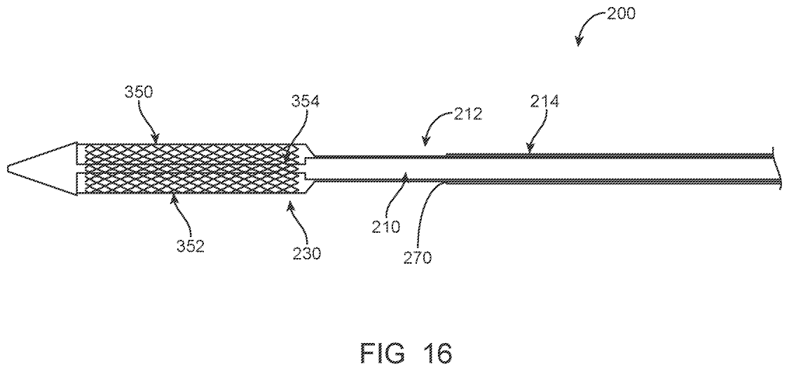

For example, FIG. 16 reflects, in simplified form, one example of the delivery device 200 loaded with a prosthetic heart valve 350 in a compressed state. With the example of FIG. 16, the prosthetic heart valve 350 includes a self-expanding stent 352 that is crimped over the inner shaft assembly 210 and secured to an optional valve retainer 354 provided with inner shaft assembly 210. The outer shaft assembly 212 is received over the inner shaft assembly 210. The capsule 230 is positioned over the prosthetic heart valve 350, maintaining the prosthetic heart valve 350 in the compressed state. Finally, the stability shaft 214 is coaxially arranged over the outer shaft assembly 212, with the distal end 270 proximally spaced from the capsule 230. Though not visible in the view of the FIG. 16, the wire(s) 216 are secured relative to the stability shaft 214 as described above.

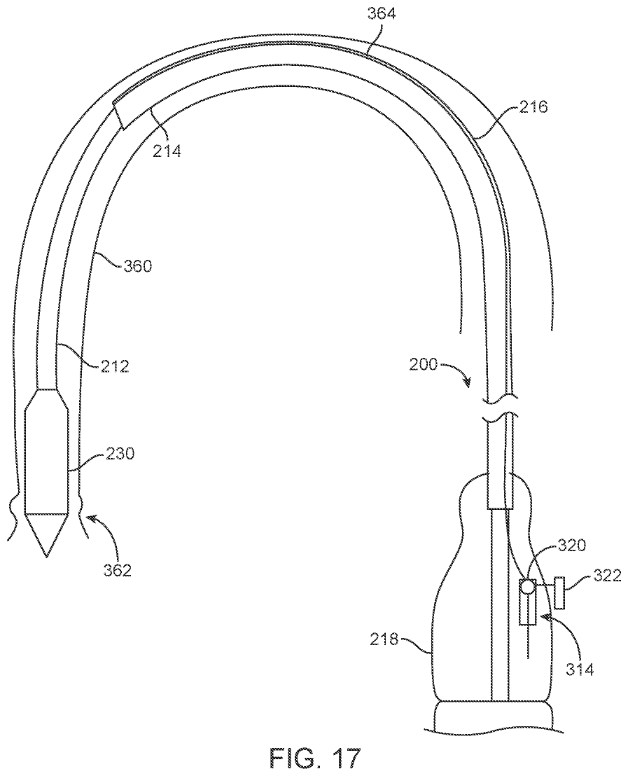

The loaded delivery device 200 is then manipulated to percutaneously deliver the prosthetic heart valve 350 (in the compressed state) through a vasculature of the patient to a target site. For example, FIG. 17 illustrates the loaded delivery device 200 having been advanced through a patient's vasculature 360 to deliver the prosthetic heart valve 350 (compressed within the capsule 230) to an aortic valve target site 362 (e.g., at the stage of delivery of FIG. 17, the capsule 230 is proximate the aortic valve target site 362). In some embodiments, an introducer (not shown) can be used to assist in establishing a portal to a bodily lumen (e.g., femoral artery) of the patient. In many instances, the vasculature 360 encountered by the delivery device 200 can be highly tortuous. For example, to attain the aortic valve target site 362, the components of the delivery device 200, including the stability shaft 214, must track along or at least partially traverse the aortic arch 364. During this tracking stage of the delivery process, then, the locking mechanism 314 is operated in the unlocked state. Under these conditions, the wire 216 is not under tension, and thus does not impede the stability shaft 214 from freely following curvatures of the aortic arch 364 for example. Thus, in the arrangement of FIG. 17, the stability shaft 214 is in contact with an outer curvature of the anatomy (i.e., anatomy of the aortic arch 364).

To deploy the prosthetic heart valve 350 (hidden) from the delivery device 200, the handle assembly 218 can be operated to proximally retract the outer shaft assembly 212 to withdraw the capsule 230 from over the prosthetic heart valve 350). The stability shaft 214 facilitates this movement (with the outer shaft assembly 212 proximally retracting relative to the stability shaft 214), acting as a distance stabilizing member to help set and maintain the distance between the handle assembly 218 and the introducer (not shown) to thereby promote accurate deployment of the prosthetic heart valve 350 at the target site 362. Under some circumstances, a user may desire to increase a bending stiffness of the stability shaft 214 prior to and during proximal retraction of the outer shaft assembly 212. For example, as the capsule 230 is retracted and exposed portions of the prosthetic heart valve 350 being to self-expand and contact native anatomy, the prosthetic heart valve 350 exerts a distal pulling force onto the delivery device 200, biasing the delivery device 200 away from the arrangement of FIG. 17. This unexpected movement can, in turn, allow the prosthetic heart valve 350 from the desired deployment position relative to the target site 362 (e.g., the distal pulling force exerted on the delivery device 200 can be sufficient to move the capsule 230/prosthetic heart valve 350 away from the aortic valve target site 362 in a direction of the left ventricle; in other words, the capsule 230/prosthetic heart valve 350 has a tendency to “dive” or move down into the annulus of the aortic valve target site 362). To address these concerns, once the capsule 230 has been located at the desired deployment position (and prior to retracting capsule 230), the locking mechanism 314 is actuated to the locked state, applying tension to the wire 216. This tension adds bending stiffness to the stability shaft 214, resulting in the stability shaft 214 (and thus the delivery device 200) becoming more rigidly “locked” to the native anatomy (e.g., contact with an outer curvature of the aortic arch 364). If desired, the user can increase tension in the wire 216, and thus bending stiffness in the stability shaft 214, by manipulating the actuator 322 to slide the lock unit 320 as described above. Enhanced bending stiffness in the stability shaft 214 can be sufficient to overcome or resist forces exerted on the delivery device 200 as the capsule 230 is subsequently retracted/the prosthetic heart valve 350 begins to self-expand. Following deployment of the prosthetic heart valve 350, the locking mechanism 314 can be returned to the unlocked state to facilitate removal of the delivery device 200 from the patient.

With this one example method, tension in the wire 216 is utilized to enhance a bending strength of the stability shaft 214 in a direction generally corresponding to outer curvature of the aortic arch 364. With optional embodiments in which two (or more) of the wires 216 are provided (e.g., commensurate with the two slots 276 shown in FIG. 12) and a separate locking mechanism is provided for each of the wires 216, a user can select the best-situation one of the wires 216 (i.e., the wire 216 that is otherwise closest to tracking along the outer curvature of the aortic arch 364) to maintain/apply tension.

The delivery device 200 can be useful with a number of other procedures or methods in addition to the transcatheter aortic valve delivery/deployment described above. Methods of the present disclosure can include any procedure in which the delivery device 200 is employed to direct any type of prosthetic heart valve to any particular target site, and during which the locking mechanism 314 is operated to selectively maintain or apply tension in the wire 216.

Although the present disclosure has been described with reference to preferred embodiments, persons skilled in the art will recognize that changes can be made in form and detail without departing from the spirit and scope of the present disclosure.

1. A transcatheter prosthetic heart valve delivery device comprising:
an outer shaft assembly defining a longitudinal axis, a proximal end, and a distal end opposite the proximal end, wherein the outer shaft assembly further defines:
a central lumen;
a slot extending through a thickness of a side wall of the outer shaft assembly, the slot being open at the proximal end;
an inner shaft assembly configured to be co-axially received within the central lumen; and
a rod sized to be slidably received within the slot, the rod defining an upper surface opposite a lower surface, wherein a thickness of the rod between the upper and lower surfaces varies along a length of the rod;
wherein the rod is selectively inserted into the slot via the proximal end such that the upper surface is radially opposite the lower surface relative to the longitudinal axis.

2. The transcatheter prosthetic heart valve delivery device of claim 1, wherein the transcatheter prosthetic heart valve delivery device is configured such that insertion of the rod into the slot increases a torqueability of the outer shaft assembly.

3. The transcatheter prosthetic heart valve delivery device of claim 1, wherein the rod defines a corrugated shape along the length thereof.

4. The transcatheter prosthetic heart valve delivery device of claim 1, wherein the lower surface is substantially flat and the upper surface has a wave-like shape.

5. The transcatheter prosthetic heart valve delivery device of claim 1, wherein the upper surface defines a series of protrusions relative to the lower surface.

6. The transcatheter prosthetic heart valve delivery device of claim 1, wherein the rod defines a first end opposite a second end, and further wherein the length of the rod is defined between the first and second ends.

7. The transcatheter prosthetic heart valve delivery device of claim 6, wherein the rod defines a first side surface opposite a second side surface each extending from the first end to the second end, and further wherein a width of the rod between the first and second side surfaces is substantially uniform along the length of the rod.

8. The transcatheter prosthetic heart valve delivery device of claim 7, wherein a major plane of each of the first and second side surfaces is substantially perpendicular to a major plane of the lower surface.

9. The transcatheter prosthetic heart valve delivery device of claim 7, wherein the rod has an elongated shape defining a central axis, and further wherein a flexibility of the rod in a first plane perpendicular to the central axis and intersecting the upper and lower surfaces is greater than a flexibility of the rod in a second plane orthogonal to the first plane and intersecting the first and second side surfaces.

10. The transcatheter prosthetic heart valve delivery device of claim 1, further comprising a handle assembly maintaining the inner and outer shaft assemblies, the handle assembly including a locking mechanism configured to selectively engage the rod to longitudinally lock the rod relative to the outer shaft assembly.

11. The transcatheter prosthetic heart valve delivery device of claim 1, wherein the inner shaft assembly includes a balloon for selectively expanding a prosthetic heart valve carried thereby.

12. A transcatheter prosthetic heart valve delivery device comprising:
a handle assembly including a locking mechanism;
an outer shaft assembly extending from the handle assembly and defining a central lumen, the outer shaft assembly including a distal region and a proximal region;
an inner shaft assembly extending from the handle assembly and configured to be co-axially received within the central lumen;
a stability shaft extending from the handle assembly and defining a central passage sized to slidably receive the proximal region of the outer shaft assembly, the stability shaft terminating at a distal end opposite the handle assembly;
a wire defining a leading section opposite a trailing section, wherein the leading section is affixed to the stability shaft proximate the distal end, and further wherein the trailing section extends proximally beyond the stability shaft and is arranged to be selectively engaged by the locking mechanism;
wherein the locking mechanism is operable to selectively lock the trailing section relative to the handle assembly such that the delivery device provides an unlocked state in which the trailing section freely slides relative to the handle assembly and a locked state in which the trailing section is locked relative to the handle assembly to maintain tension in the wire.

13. The transcatheter prosthetic heart valve delivery device of claim 12, wherein the delivery device is configured such that in the locked state, tension in the wire generates a bending stiffness in the stability shaft.

14. The transcatheter prosthetic heart valve delivery device of claim 12, wherein the stability shaft includes a wall, and further wherein a slot is defined in a thickness of the wall for receiving the wire.

15. The transcatheter prosthetic heart valve delivery device of claim 14, wherein the wire defines an intermediate section between the leading and trailing sections, and further wherein the intermediate section extends within the slot.

16. The transcatheter prosthetic heart valve delivery device of claim 15, wherein the intermediate section is free of direct, physical fixation to the stability shaft.

17. The transcatheter prosthetic heart valve delivery device of claim 12, wherein the leading section is laminated to the stability shaft.

18. The transcatheter prosthetic heart valve delivery device of claim 12, wherein the wire is a first wire and the delivery device further comprises a second wire defining a leading section opposite a trailing section, wherein the leading section of the second wire is secured to the stability shaft proximate the distal end, and further wherein the trailing section of the second wire extends proximally beyond the stability shaft and is arranged to be selectively engaged by the locking mechanism.

19. The transcatheter prosthetic heart valve delivery device of claim 18, wherein relative to a circumference of the stability shaft, the first wire is circumferentially spaced from the second wire by approximately 90 degrees.

20. The transcatheter prosthetic heart valve delivery device of claim 12, wherein a length of the stability shaft is less than a length of the outer shaft assembly.

21. The transcatheter prosthetic heart valve delivery device of claim 12, wherein the outer shaft assembly includes a capsule for maintaining a prosthetic heart valve carried by the inner shaft assembly in a delivery state, and further wherein the distal end of the stability shaft is proximal the capsule.
